(19) United States
(12) Patent Application Publication
(10) Pub. No.: US 2024/0075273 A1
(43) Pub. Date: Mar. 7, 2024

(54) NEEDLELESS CONNECTOR

(71) Applicant: CareFusion 303, Inc., San Diego, CA (US)

(72) Inventors: Jason Andrew Wine, Brea, CA (US); George Mansour, Diamond Bar, CA (US); Bin Wang, Sandy, UT (US); Amarsinh Deeliprao Jadhav, Bangalore (IN); Mohamed Shafiq, Bangalore (IN); Kowshika K, Tirupur (IN)

(21) Appl. No.: 17/939,452

(22) Filed: Sep. 7, 2022

(51) Int. Cl. A61M 39/10; A61M 39/26

(52) U.S. Cl. CPC A61M 39/10; A61M 39/26

(57) ABSTRACT

Needleless connectors are described herein. A needleless connector includes a housing and a flexible valve element. The housing includes a cavity, a proximal fluid port in fluid communication with the cavity, and a distal fluid port in fluid communication with the cavity. The flexible valve element is disposed within the cavity. The flexible valve element can selectively permit flow between the proximal fluid port and the distal fluid port.

### FIELD OF THE INVENTION

The present disclosure generally relates to connectors, and, in particular, to needleless connectors.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. Certain needleless connectors may be used in an IV set and may have a self-sealing port to prevent leakage of fluid when a mating medical implement is decoupled from such a needleless connector. Additionally, a needleless connector may include a mechanical valve, for example, a collapsible valve comprising a flexible material for providing the self-sealing port and controlling the flow of fluid within the IV set.

In some applications, needleless connectors may be difficult to connect, prime, flush, and/or disconnect without forming droplets.

### SUMMARY

The disclosed subject matter relates to connectors having improved valve elements and housings. In certain embodiments, a needleless connector is disclosed that comprises a housing comprising a cavity, a proximal fluid port in fluid communication with the cavity, and a distal fluid port in fluid communication with the cavity; and a flexible valve element disposed within the cavity to selectively permit flow between the proximal fluid port and the distal fluid port.

In certain embodiments, a needleless connector is disclosed that comprises a housing comprising: a proximal portion; a distal portion, wherein the proximal portion and the distal portion are coupled together to cooperatively define a cavity; a proximal fluid port in fluid communication with the cavity; and a distal fluid port in fluid communication with the cavity; and a flexible valve element comprising: a valve diaphragm, and the valve diaphragm is configured to expand when a luer is inserted into the proximal fluid port and retract when the luer is removed from the proximal fluid port; a cone coupled to the valve diaphragm and extending toward the proximal fluid port; a retention edge, wherein at least a portion of the retention edge is captured within the housing; and a valve tail extending into the cavity, wherein the valve tail deflects to selectively permit flow between the proximal fluid port and the distal fluid port.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a cross-sectional view of a needleless connector, in accordance with various aspects of the present disclosure.

FIG. 2 is a side elevation view of a flexible valve element, in accordance with various aspects of the present disclosure.

FIG. 3 is a front elevation view of a flexible valve element, in accordance with various aspects of the present disclosure.

FIG. 4 is a partial cross-sectional view of a needleless connector with the flexible valve element in a sealing position, in accordance with various aspects of the present disclosure.

FIG. 5 is a partial cross-sectional view of a needleless connector with the flexible valve element in a flow position, in accordance with various aspects of the present disclosure.

FIG. 6 is a partial cross-sectional view of a flexible valve element, in accordance with various aspects of the present disclosure.

FIG. 7 is a partial cross-sectional view of a flexible valve element, in accordance with various aspects of the present disclosure.

FIG. 8 is a perspective view of a flexible valve element, in accordance with various aspects of the present disclosure.

FIG. 9 is a cross-sectional view of a flexible valve element, in accordance with various aspects of the present disclosure.

FIG. 10 is a top view of a flexible valve element, in accordance with various aspects of the present disclosure.

FIG. 11 is a partial cross-sectional view of a needleless connector, in accordance with various aspects of the present disclosure.

FIG. 12 is a partial cross-sectional view of a needleless connector, in accordance with various aspects of the present disclosure.

FIG. 13 is a perspective cross-sectional view of a housing, in accordance with various aspects of the present disclosure.

FIG. 14 is a detail cross-sectional view of a housing, in accordance with various aspects of the present disclosure.

FIG. 15 is an elevation cross-sectional view of a housing, in accordance with various aspects of the present disclosure.

FIG. 16 is a perspective view of a housing, in accordance with various aspects of the present disclosure.

FIG. 17 is a cross-sectional view of a needleless connector, in accordance with various aspects of the present disclosure.

FIG. 18 is a cross-sectional view of a needleless connector, in accordance with various aspects of the present disclosure.

FIG. 19 is a cross-sectional view of a needleless connector, in accordance with various aspects of the present disclosure.

FIG. 20 is a cross-sectional view of a needleless connector, in accordance with various aspects of the present disclosure.

FIG. 21 is a cross-sectional view of a needleless connector, in accordance with various aspects of the present disclosure.

FIG. 22 is a detail cross-sectional view of a housing, in accordance with various aspects of the present disclosure.

FIG. 23 is a perspective view of a housing portion, in accordance with various aspects of the present disclosure.

FIG. 24 is a cross-sectional view of a needleless connector, in accordance with various aspects of the present disclosure.

FIG. 25 is a perspective view of a housing, in accordance with various aspects of the present disclosure.

FIG. 26 is a cross-sectional view of a needleless connector, in accordance with various aspects of the present disclosure.

FIG. 27 is a perspective view of a housing portion, in accordance with various aspects of the present disclosure.

FIG. 28 is a cross-sectional view of a needleless connector, in accordance with various aspects of the present disclosure.

FIG. 29 is a cross-sectional view of a needleless connector, in accordance with various aspects of the present disclosure.

FIG. 30 is a perspective view of a collar, in accordance with various aspects of the present disclosure.

FIG. 31 is a cross-sectional view of a flexible valve element, in accordance with various aspects of the present disclosure.

FIG. 32 is a cross-sectional view of a needleless connector, in accordance with various aspects of the present disclosure.

FIG. 33 is a partial cross-sectional view of a collar, in accordance with various aspects of the present disclosure.

FIG. 34 is a partial cross-sectional view of a housing portion, in accordance with various aspects of the present disclosure.

FIG. 35 is a cross-sectional view of a flexible valve element, in accordance with various aspects of the present disclosure.

FIG. 36 is a cross-sectional view of a needleless connector, in accordance with various aspects of the present disclosure.

FIG. 37 is a detail cross-sectional view of a needleless connector, in accordance with various aspects of the present disclosure.

FIG. 38 is a perspective view of a collar, in accordance with various aspects of the present disclosure.

FIG. 39 is a partial cross-sectional view of a collar, in accordance with various aspects of the present disclosure.

FIG. 40 is a cross-sectional view of a needleless connector, in accordance with various aspects of the present disclosure.

FIG. 41 is a cross-sectional view of a needleless connector, in accordance with various aspects of the present disclosure.

FIG. 42 is a cross-sectional view of a needleless connector, in accordance with various aspects of the present disclosure.

FIG. 43 is a detail view of a housing portion, in accordance with various aspects of the present disclosure.

FIG. 44 is a detail view of a housing portion, in accordance with various aspects of the present disclosure.

FIG. 45 is a detail cross-sectional view of a needleless connector, in accordance with various aspects of the present disclosure.

### DETAILED DESCRIPTION

The disclosed needleless connector incorporates various features to facilitate the connection, priming, flushing, and/or disconnection of the connector. Features can include, but are not limited to features of the valve tail, features of the valve diaphragm, and features of the housing design. By providing a needleless connector with one or more of these features, the needleless connector can provide improved connection, priming, flushing, and/or disconnection operation compared to conventional needleless connectors.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to the administration of medical fluid to a patient by a medical practitioner using the disclosed needleless connector, it is to be understood that this description is only an example of usage and does not limit the scope of the claims. Various aspects of the disclosed needleless connectors may be used in any application where it is desirable to prevent the transfer of bacteria, microorganisms, and other pathogens while permitting the draw of blood and other fluids from the patient.

The disclosed needleless connector overcomes several challenges discovered with respect to certain conventional connectors. One challenge with certain conventional needleless self-sealing connectors is that certain conventional needleless connectors may be difficult to connect, which may increase the time a clinician must spend connecting an IV set. Another challenge with certain conventional needleless self-sealing connectors is that certain conventional connectors may require a large volume for priming and flushing, which may increase the amount of fluid administered or lead to inaccurate administration of fluids. Further, another challenge with certain conventional needleless self-sealing connectors is that certain conventional may create large droplets upon disconnection. Additionally, another challenge with certain conventional needleless self-sealing connectors is that certain conventional connectors may include a valve element that is not resilient or easy to manufacture.

Therefore, in accordance with the present disclosure, it is advantageous to provide a needleless connector as described herein that can facilitate connections, priming, and disconnections. Further, it is advantageous to provide a needleless connector as described herein that can include features that are resilient and readily manufactured.

An example of a needleless connector that provides improved connection, priming, flushing, and/or disconnection operation compared to conventional needleless connectors is now described.

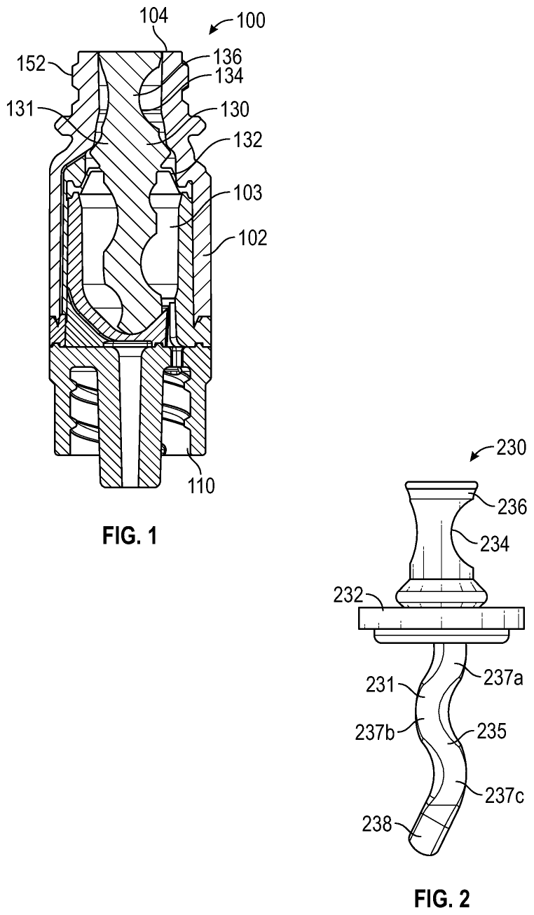

FIG. 1 is a cross-sectional view of a needleless connector 100 in accordance with various aspects of the present disclosure. In the depicted example, the needleless connector 100 is a self-sealing port that provides IV access to a patient while further preventing leakage of fluid when the mating medical implement is decoupled from the needleless connector 100. As illustrated, fluid flow from an IV set can be introduced into a patient via a proximal fluid port 104, through a housing 102, through the distal fluid port 110 and to a patient via a patient tubing. Further, in some embodiments, blood and other fluids from the patient can be drawn from the distal fluid port 110 through the housing 102 to the proximal fluid port 104. As shown, the proximal fluid port 104 and the distal fluid port 110 can include any suitable fitting, including, but not limited to Luer fittings. In the depicted example, the proximal fluid port 104 is shown as a female Luer fitting and the distal fluid port 110 is shown as a male Luer fitting.

As illustrated, the housing 102 of the needleless connector 100 contains the flexible valve element 130 within the cavity 103 of the housing 102. In the depicted example, the proximal fluid port 104 is in fluid communication with the cavity 103 of the housing 102. The proximal fluid port 104 can be threaded to facilitate connections with mating medical implements.

As illustrated, the flexible valve element 130 can permit or restrict flow through the proximal fluid port 104 by selectively sealing against the inner cavity 103 of the housing 102. The flexible valve element 130 can have a body 131 with a sealing portion 132 disposed toward the proximal end 136 of the flexible valve element 130. The sealing portion 132 can further include a cut portion 134 to facilitate flow past the flexible valve element 130 by readily deforming upon insertion of a mating medical implement. The body 131 of the flexible valve element 130 can be formed from silicone or other elastomeric materials to resiliently deform and reform to allow selective sealing of the proximal fluid port 104. The distal fluid port 110 can be in fluid communication with the cavity 103 generally. The distal fluid port 110 can be coupled to the patient tubing.

In the depicted example, the flexible valve element 130 forms a seal to prohibit the flow of fluid through the proximal fluid port 104 and the needleless connector 100 generally when a mating medical implement is not connected to the proximal fluid port 104, thereby preventing leakage. Accordingly, when the needleless connector 100 is not being accessed, the sealing portion 132 and the flexible body 131 of the flexible valve element 130 generally seal against the interior cavity 103 to prevent fluid flow therethrough.

During operation, a mating medical implement can be attached to the needleless connector 100. The medical implement can be used to introduce a fluid or medicine to the patient or to draw blood or other fluids from the patient via the needleless connector 100. The medical implement can be connected to the proximal fluid port 104 via a threaded connection 152.

During operation, the male fitting of the medical implement can be introduced into the cavity 103 of the housing 102. Upon introduction of the male fitting into the cavity 103, the flexible valve element 130 can be sufficiently elastic to deform or bend out of sealing engagement with the cavity 103 to permit fluid flow between the needleless connector 100 and the medical implement 150. The flexible valve element 130 can return to its original shape upon disconnection of the male fitting.

In the depicted example, the needleless connector 100 can be a positive displacement device. For example, when a new connection is made at the proximal fluid port 104, the volume of the internal cavity 103 is reduced, and the needleless connector 100 draws fluid in from the proximal fluid port 104 or the distal fluid port 110. Accordingly, when disconnection is made at the proximal fluid port 104, the needleless connector 100 expels fluid from the cavity 103, effectively flushing the needleless connector 100.

During operation, when fluid is introduced from the medical implement into the patient such as when introducing saline or medicine into a patient, fluid can flow from the proximal fluid port 104 to the distal fluid port 110 and into the patient tubing. In the depicted example, the flow is directed from the medical implement towards the cavity 103. The bent or deformed flexible valve element 130 allows for the flow to pass beyond the proximal fluid port 104 through the distal fluid port 110 to the patient tubing.

In some applications, when fluid is drawn from the patient, such as when blood is drawn, the fluid can flow from the distal fluid port 110 to the proximal fluid port 104 and further to the medical implement. In the depicted example, drawn fluid from the patient is directed from the distal fluid port 110 past the bent or deformed flexible valve element 130 to allow the flow to continue to the proximal fluid port 104.

As described herein, one or more elements of the needleless connector 100 can be modified to facilitate improved connections, priming, and disconnections compared to certain conventional connectors.

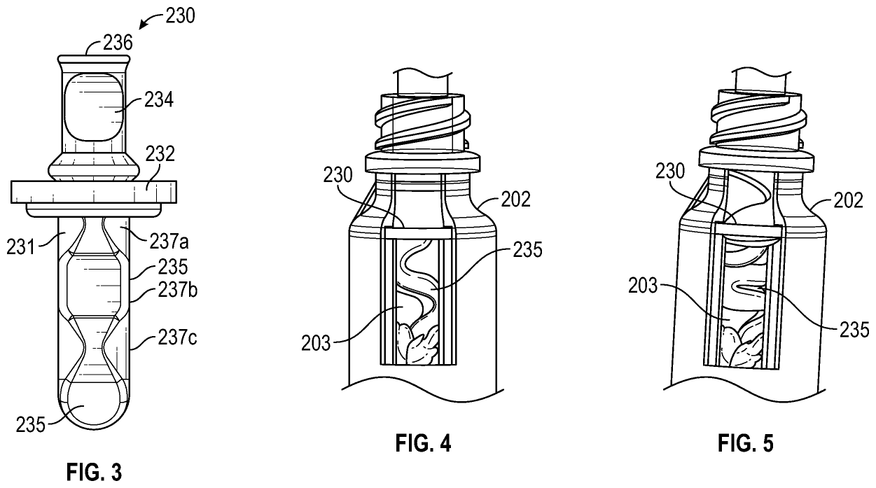

FIG. 2 is a side elevation view of a flexible valve element 230, in accordance with various aspects of the present disclosure. FIG. 3 is a front elevation view of a flexible valve element 230, in accordance with various aspects of the present disclosure. FIG. 4 is a partial cross-sectional view of a needleless connector 200 with the flexible valve element 230 in a sealing position, in accordance with various aspects of the present disclosure. FIG. 5 is a partial cross-sectional view of a needleless connector 200 with the flexible valve element 230 in a flow position, in accordance with various aspects of the present disclosure. In some embodiments, the needleless connector 200 can include features that are similar to needleless connector 100. Accordingly, certain features of the needleless connector 200 can be referred to with similar reference numerals. With reference to FIGS. 2-5, the flexible valve element 230 can include features that allow a clinician to easily make a connection with the needleless connector 200.

As described with respect to needleless connector 100, the flexible valve element 230 can permit or restrict flow through the needleless connector 200. During operation, the flexible valve element 230 deforms to permit flow through the needleless connector 200. In the depicted example, the flexible valve element 230 includes an elongated tail 235 to control the amount of force required to sufficiently deform the flexible valve element 230 and permit flow through the needleless connector 200.

In the depicted example, the flexible valve element 230 includes an elongated tail 235 with a reduced spring rate to reduce the insertion force required to sufficiently deform the flexible valve element 230 and permit flow through the needleless connector 200. In some embodiments, the elongated tail 235 of the flexible valve element 230 is longer than the tail of the flexible valve element 230. Accordingly, in some applications, the elongated tail 235 of the flexible valve element 230 can allow for a lower spring force over a longer distance or length, allowing for easier insertion. Further, the extended length and lower spring force of the elongated tail 235 can allow more deterministic and/or rapid closure of the needleless connector 200 during the removal of a mating medical implement.

In some embodiments, the elongated length of the elongated tail 235 can allow for a larger amount of deflection of the elongated tail 235 in an unactuated state, increasing preload of the flexible valve element 230. In some embodiments, the elongated tail 235 can include one or more bends 237a, 237b, 237c to allow for a longer elongated tail 235 to be disposed within the cavity 203 of the housing 202. Further, in some applications, the bends 237a, 237b, 237c can facilitate bending or deformation of the elongated tail 235. In some applications, the cavity 203 can be enlarged compared to cavity 103 to facilitate deflection of the elongated tail 235 without prematurely “bottoming out” during the insertion of the mating medical implement.

Advantageously, the configuration of the elongated tail 235 and/or the enlarged cavity 203 can allow for a reduced connection force, reduced droplet size, and/or reduced droplet splash. For example, the elongated tail 235 can allow for a more deterministic closure of the needleless connector 200 upon removal of the mating medical instrument, allowing for reduced droplet size. However, the response time of the elongate tail 235 may be rapid enough to maintain contact with the mating medical instrument (e.g., syringe) during rapid removal. Advantageously, the rapid response of elongate tail 235 can prevent the build of fluid volume in front of the valve face, preventing droplet splash.

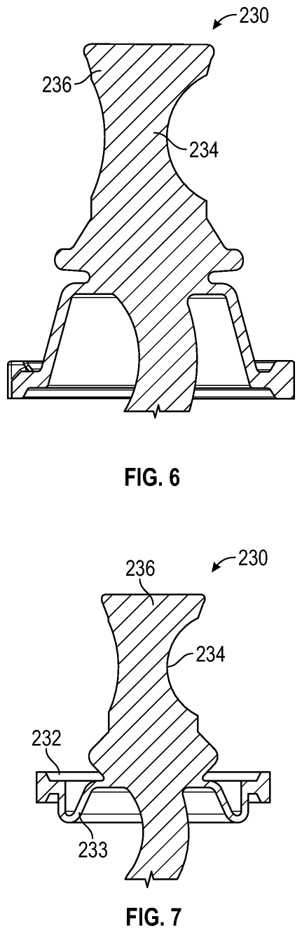

FIG. 6 is a partial cross-sectional view of a flexible valve element 230, in accordance with various aspects of the present disclosure. With reference to FIG. 6, in some applications, the proximal end 236 of the flexible valve element 230 can have a generally cone shape to permit sealing and/or engagement with the respective housing. In some embodiments, the cone shape of the proximal end 236 can be enlarged compared to certain conventional needleless valve elements to improve sealing performance.

FIG. 7 is a partial cross-sectional view of a flexible valve element 230, in accordance with various aspects of the present disclosure. With reference to FIG. 7, in some embodiments, the sealing portion or diaphragm 232 of the flexible valve element 230 can be configured to displace a low volume of fluid during connection and disconnection of the needleless connector 200. During operation, the diaphragm 232 can expand when a mating medical instrument is inserted and can retract when the mating medical instrument is removed. The removal of the mating medical instrument can cause a positive fluid displacement during disconnection. In some embodiments, the structure and/or configuration of the diaphragm can result in a lower volume displacement than during the disconnection of the needleless connector 100.

In some embodiments, the diaphragm body 233 can be extended to increase the movement path length of the diaphragm 232 during the insertion of a mating medical instrument. Advantageously, by extending the length of travel of the diaphragm 232 by elongating the diaphragm body 233, strain in the diaphragm body 233 can be reduced during insertion, increasing resilience of the flexible valve element. In some applications, the diaphragm 232 can be more resistant to tearing compared to certain conventional valve elements.

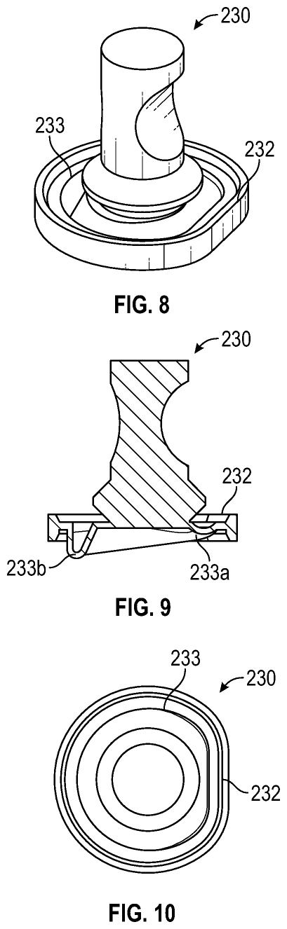

FIG. 8 is a perspective view of a flexible valve element 230, in accordance with various aspects of the present disclosure. FIG. 9 is a cross-sectional view of a flexible valve element 230, in accordance with various aspects of the present disclosure. FIG. 10 is a top view of a flexible valve element 230, in accordance with various aspects of the present disclosure. With reference to FIGS. 8-10, the diaphragm 232 can include additional features to reduce strain during operation. For example, the diaphragm 282 can be designed in an asymmetrical shape. As illustrated, the diaphragm 232 can be configured to be a “D” shape to promote the diaphragm body 233 to extend or move in a certain path to minimize stress on the diaphragm 232.

In some applications, the diaphragm body 233 can be extended asymmetrically to increase the movement path length of one side of the diaphragm 232 during the insertion of a mating medical instrument. For example, one portion of the diaphragm body 233b can define a longer path length than a shorter side of the diaphragm body 233a. Advantageously, by extending the length of travel of the diaphragm 232 on one side by elongating the diaphragm body 233b, strain in the diaphragm body 233 can be reduced during insertion, increasing resilience of the flexible valve element. Further, by elongating the diaphragm body 233b, the flexible valve element 230 may be biased or otherwise configured to deform or deflect in a certain, predictable manner. Further, in some embodiments, the elongated length of the diaphragm body 233b can correspond to the rounded portion of the “D” shape of the diaphragm body 232.

FIG. 11 is a partial cross-sectional view of a needleless connector 200, in accordance with various aspects of the present disclosure. In the depicted example, the housing 202 can retain or capture the flexible valve element 230 relative to the housing 202 to maintain sterility of the fluid path and maintain the position of the diaphragm 232 relative to the housing 202. In some embodiments, the housing 202 can include a tapered channel 205 to capture and retain the diaphragm 232 of the flexible valve element 230. Optionally, a portion of the diaphragm 232 can be deformed within the tapered channel 205, allowing the diaphragm 232 and the flexible valve element 230 generally to be captured relative to the housing 202.

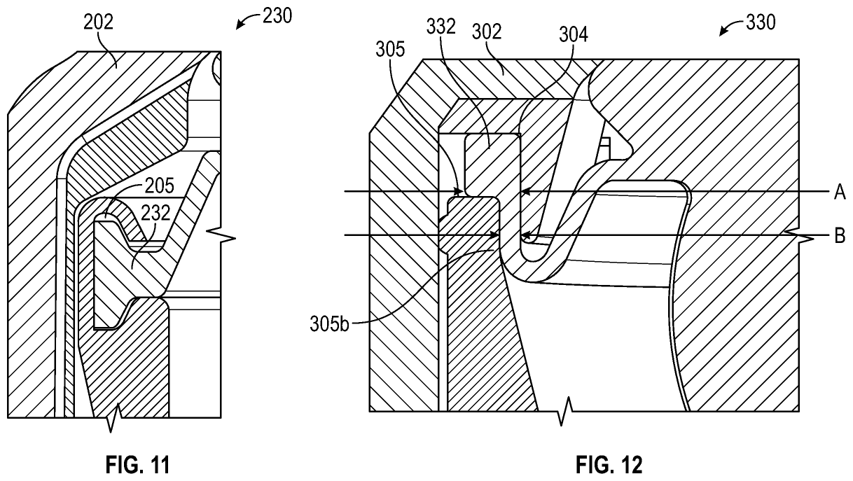

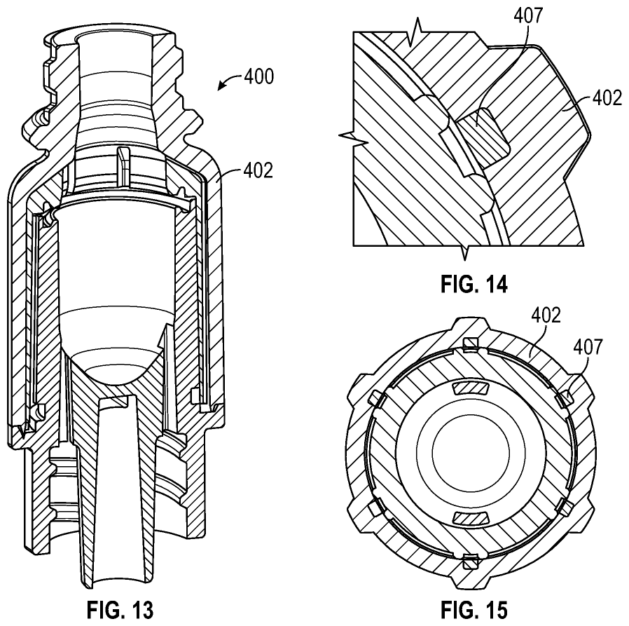

FIG. 12 is a partial cross-sectional view of a needleless connector 200, in accordance with various aspects of the present disclosure. In some embodiments, the needleless connector 300 can include features that are similar to needleless connector 200. Accordingly, certain features of the needleless connector 300 can be referred to with similar reference numerals.

Similar to needleless connector 200, the housing 302 can retain or capture a flexible valve element 330 relative to the housing 302. In the depicted example, the housing 302 can include a channel 305 to capture and retain the diaphragm 332 of the flexible valve element 330. As illustrated, the channel 305 can include a wide portion 305a with a width A and a narrow portion 305b with a width B. The geometry of the channel 305 can allow for a wide portion of the diaphragm 332 to be disposed within the wide portion 305a, while the narrow portion 305b prevents the diaphragm 332 from passing through the narrow portion 305b and prevents the diaphragm 332 from pulling inward during the insertion of a mating medical implement.

FIG. 13 is a perspective cross-sectional view of a housing 402, in accordance with various aspects of the present disclosure. FIG. 13 is a detail cross-sectional view of a housing 402, in accordance with various aspects of the present disclosure. FIG. 15 is an elevation cross-sectional view of a housing 402, in accordance with various aspects of the present disclosure. In some embodiments, the needleless connector 400 can include features that are similar to needleless connector 100. Accordingly, certain features of the needleless connector 400 can be referred to with similar reference numerals. In the depicted example, the housing 402 can define one or more flow channels 407 to facilitate flushing of the needleless connector 400. Advantageously, flow channels 407 formed in the housing 402 can allow for efficient flushing of the needleless connector 400. In some embodiments, the housing 402 is a two-part housing that defines the flow channels 407 between the two parts of the housing 402. The flow channels 407 can be contained by crush ribs.

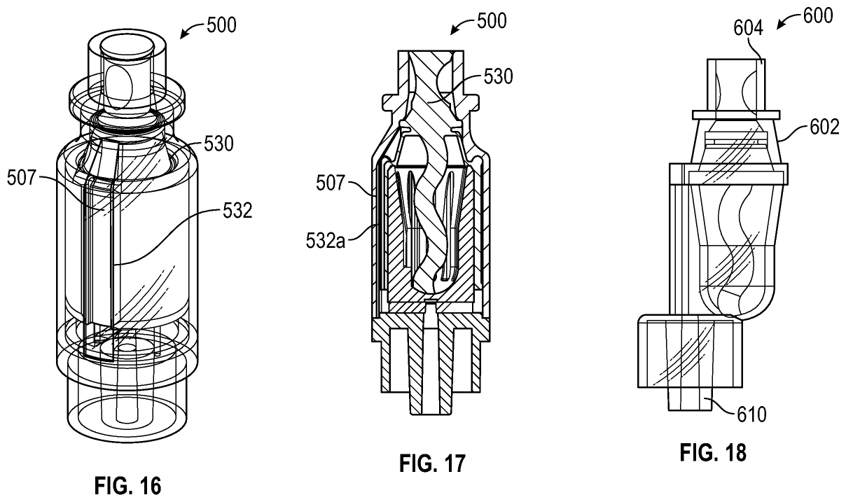

FIG. 16 is a perspective view of a housing 502, in accordance with various aspects of the present disclosure. FIG. 17 is a cross-sectional view of a needleless connector 500, in accordance with various aspects of the present disclosure. In some embodiments, the needleless connector 500 can include features that are similar to needleless connector 100. Accordingly, certain features of the needleless connector 500 can be referred to with similar reference numerals. In some embodiments, the diaphragm 532 of the flexible valve element 520 can include a shroud 532a that extends axially along the length of the housing 502. In some embodiments, a silicone wall can seal a flow channel 507 from other portions of the housing 502.

FIG. 18 is a cross-sectional view of a needleless connector 600, in accordance with various aspects of the present disclosure. In some embodiments, the needleless connector 600 can include features that are similar to needleless connector 100. Accordingly, certain features of the needleless connector 600 can be referred to with similar reference numerals. In the depicted example, the proximal fluid port 604 and the distal fluid port 610 of the housing 620 can be offset relative to each other. As illustrated, the axis of the proximal fluid port 604 is laterally offset relative to the axis of the distal fluid port 610.

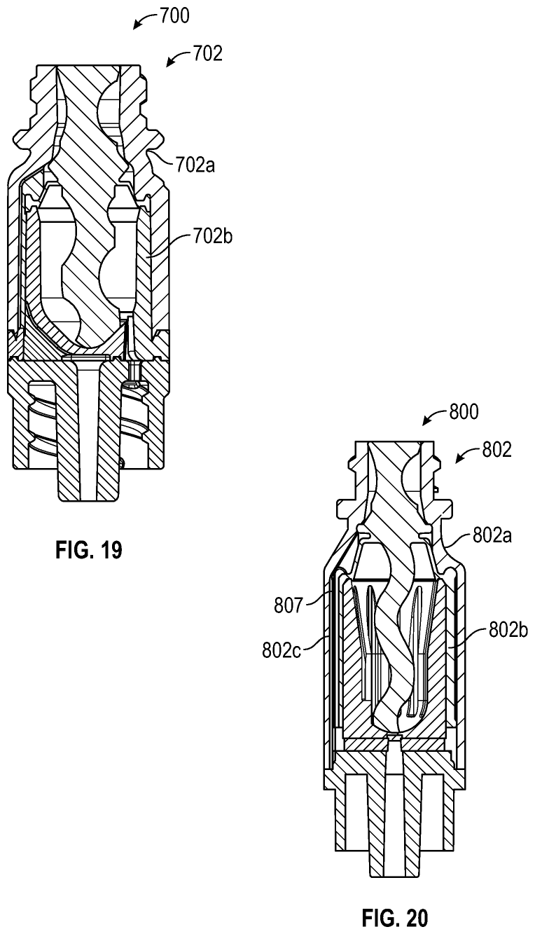

FIG. 19 is a cross-sectional view of a needleless connector 700, in accordance with various aspects of the present disclosure. In some embodiments, the needleless connector 700 can include features that are similar to needleless connector 100. Accordingly, certain features of the needleless connector 700 can be referred to with similar reference numerals. In the depicted example, the housing 702 can be a two-part housing that is snap or press fit together. In some embodiments, an upper portion 702a can be coupled to a lower portion 702b to define the housing. The upper portion 702a can be coupled to the lower portion 702b with snap fittings. Optionally, certain edges of the upper portion 702a and lower portion 702b can include press fittings to allow for fluid sealing.

FIG. 20 is a cross-sectional view of a needleless connector 800, in accordance with various aspects of the present disclosure. In some embodiments, the needleless connector 800 can include features that are similar to needleless connector 700. Accordingly, certain features of the needleless connector 800 can be referred to with similar reference numerals. Similar to needleless connector 700, the housing 802 can have a multi-part construction. In some embodiments, a flow channel 807 can be defined by a silicone or soft plastic material. The material defining the flow channel 807 can be over molded and/or press fit to allow for a seal. Advantageously, the softer material of the silicone or plastic material can absorb less energy during an ultrasonic welding process.

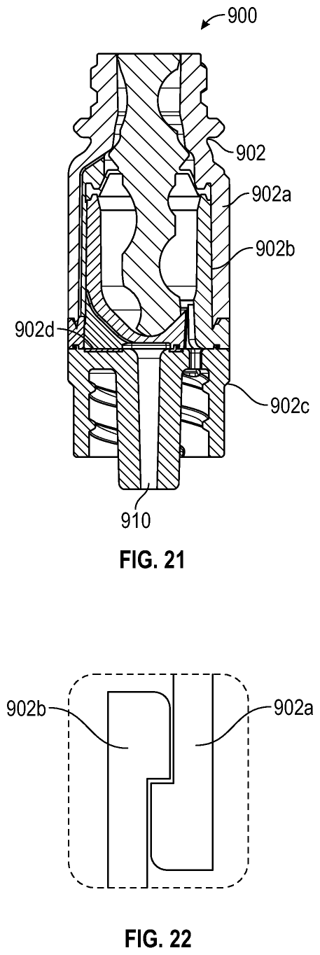

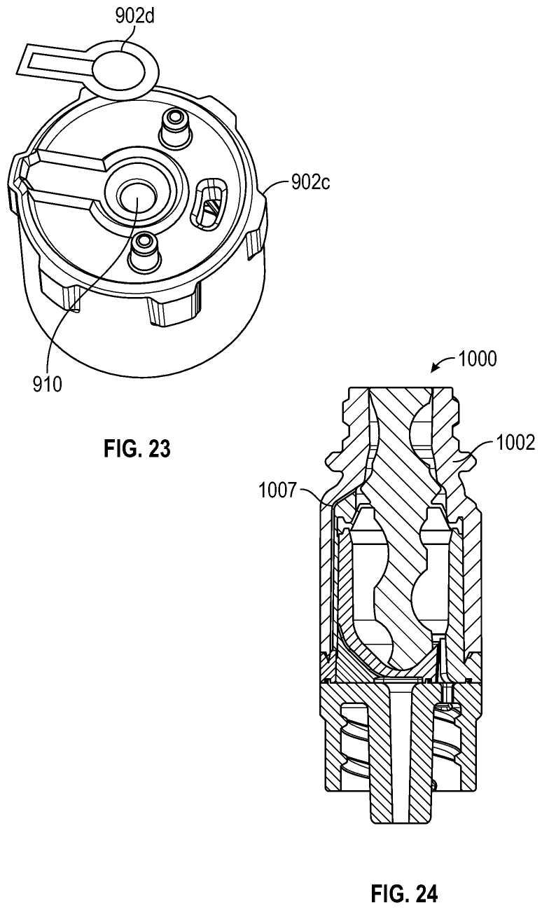

FIG. 21 is a cross-sectional view of a needleless connector 900, in accordance with various aspects of the present disclosure. FIG. 22 is a detail cross-sectional view of a housing 902, in accordance with various aspects of the present disclosure. FIG. 23 is a perspective view of a housing portion or base 902c and 902d, in accordance with various aspects of the present disclosure. In some embodiments, the needleless connector 900 can include features that are similar to needleless connector 800. Accordingly, certain features of the needleless connector 900 can be referred to with similar reference numerals. Similar to needleless connector 800, the housing 902 can have a multi-part construction.

In some embodiments, an upper portion 902a can be coupled to a lower portion 902b to define the housing 902. The upper portion 902a can be coupled to the lower portion 902b with snap fittings, as shown in FIG. 22. As illustrated, the needleless connector 900 can include an o-ring 902d disposed between the base 902c and other portions of the housing 902 to seal the fluid path within the housing and to a distal fluid port 910. In some embodiments, components of the housing 902 can be ultrasonically welded and/or glued. Advantageously, the construction of the needleless connector 900 can reduce the complexity of the assembly.

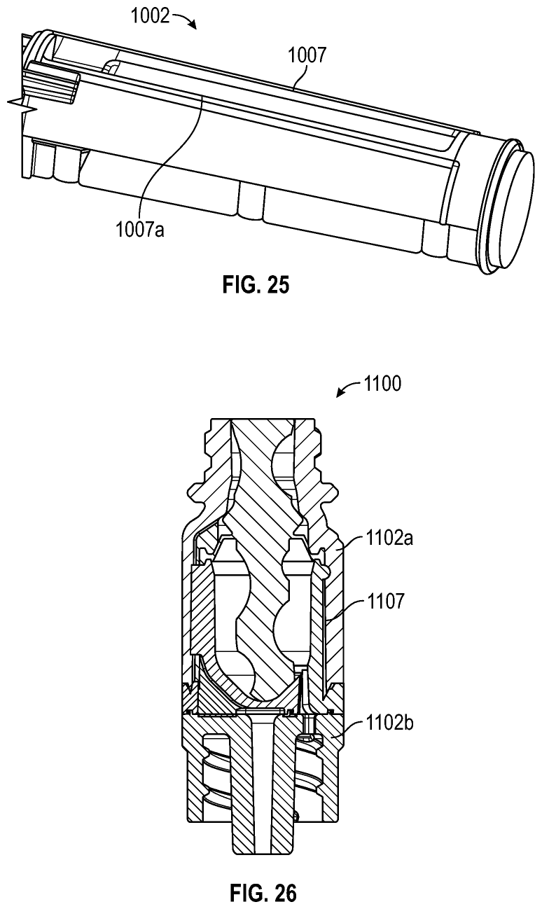

FIG. 24 is a cross-sectional view of a needleless connector 1000, in accordance with various aspects of the present disclosure. FIG. 25 is a perspective view of a housing 1002, in accordance with various aspects of the present disclosure. In some embodiments, the needleless connector 1000 can include features that are similar to needleless connector 100. Accordingly, certain features of the needleless connector 1000 can be referred to with similar reference numerals.

In the depicted example, the housing 1002 can define one or more flow channels 1007 to facilitate flushing of the needleless connector 1000. Advantageously, flow channels 1007 formed in the housing 1002 can allow for efficient flushing of the needleless connector 1000. In some embodiments, the housing 1002 is a two-part housing that defines the flow channels 1007 between the two parts of the housing 1002. The flow channels 1007 can be contained by crush ribs 1007a to prevent the weeping of fluid to other portions or outside of the housing 1002.

As illustrated, the crush ribs 1007a can be molded on a portion of the housing 1002 and engage with (via interference fit) with the interior geometry of the cap of the housing 1002 to form liquid-tight barriers. The housing 1002 can include two crush ribs 1007a to define a main flow channel 1007. The housing 1002 can further include a crush rib 1007a around the edge to further prevent weeping of fluid.

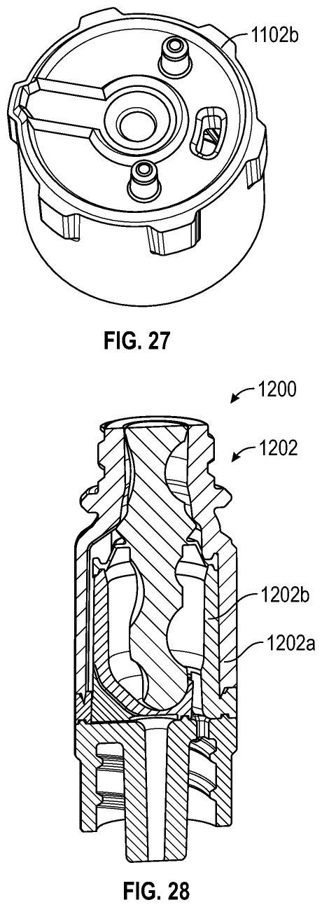

FIG. 26 is a cross-sectional view of a needleless connector 1100, in accordance with various aspects of the present disclosure. FIG. 27 is a perspective view of a housing portion 1102, in accordance with various aspects of the present disclosure. In some embodiments, the needleless connector 1100 can include features that are similar to needleless connector 100. Accordingly, certain features of the needleless connector 1100 can be referred to with similar reference numerals.

In the depicted example, the housing 1102 can define one or more flow channels 1107 to facilitate flushing of the needleless connector 1100. Advantageously, flow channels 1107 can be configured to minimize priming and flush volume. As illustrated, the volume of the flow channel 1107 disposed on the base 1102b can be reduced in volume to reduce fluid volume during priming and/or flushing.

FIG. 28 is a cross-sectional view of a needleless connector 1200, in accordance with various aspects of the present disclosure. In some embodiments, the needleless connector 1200 can include features that are similar to needleless connector 100. Accordingly, certain features of the needleless connector 1200 can be referred to with similar reference numerals. In the depicted example, the housing 1202 can provide a clearance fit between an outer portion of the housing 1202a and a collar 1202b of the housing 1202. Advantageously, by providing a clearance fit between the housing 1202 and the collar 1202b, welding or additional process steps can be reduced.

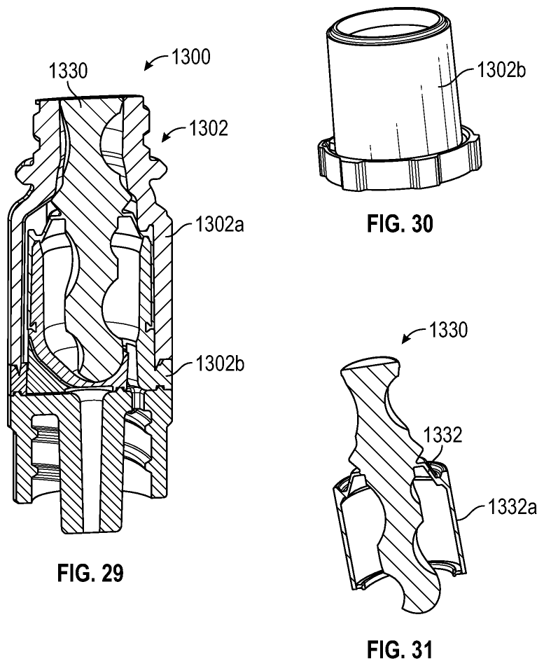

FIG. 29 is a cross-sectional view of a needleless connector 1300, in accordance with various aspects of the present disclosure. FIG. 30 is a perspective view of a collar 1302b, in accordance with various aspects of the present disclosure. FIG. 31 is a cross-sectional view of a flexible valve element 1330, in accordance with various aspects of the present disclosure. In some embodiments, the needleless connector 1300 can include features that are similar to needleless connector 100. Accordingly, certain features of the needleless connector 1300 can be referred to with similar reference numerals. In the depicted example, the housing 1302 can provide clearance between an outer portion of the housing 1302a and a collar 1302b of the housing 1302. In some embodiments, an elongated septum 1332a extending from the diaphragm 1332 of the flexible valve element 1330 can extend into the clearance defined between the housing 1302a and the collar 1302b. As illustrated, the diameter of the 1302b can be reduced to permit clearance for the septum 1332a within the housing 1302.

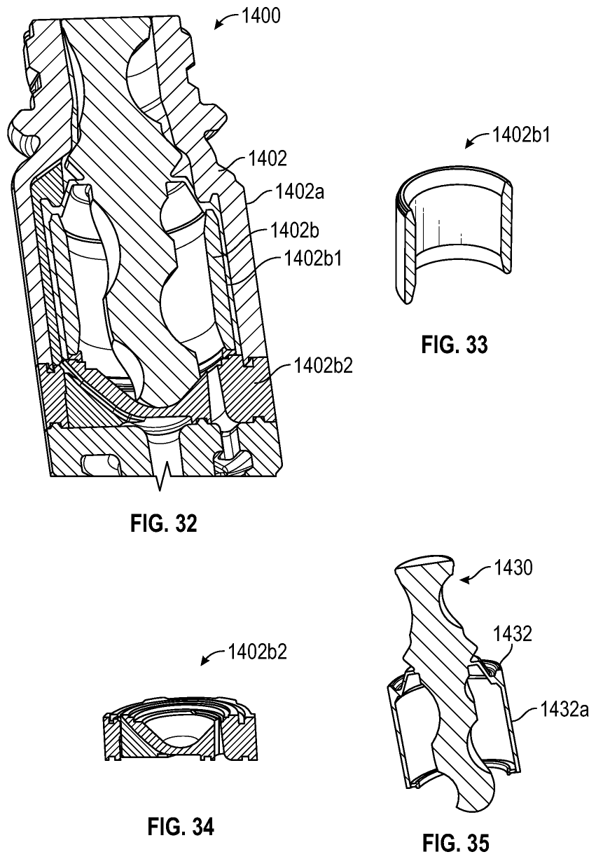

FIG. 32 is a cross-sectional view of a needleless connector 1400, in accordance with various aspects of the present disclosure. FIG. 33 is a partial cross-sectional view of a collar 1402b1, in accordance with various aspects of the present disclosure. FIG. 34 is a partial cross-sectional view of a housing portion, in accordance with various aspects of the present disclosure. FIG. 35 is a cross-sectional view of a flexible valve element 1430, in accordance with various aspects of the present disclosure. In some embodiments, the needleless connector 1400 can include features that are similar to needleless connector 1300. Accordingly, certain features of the needleless connector 1400 can be referred to with similar reference numerals. Similar to the needleless connector 1300, the housing 1402 can provide clearance between an outer portion of the housing 1402a and a collar 1402b of the housing 1402. In some embodiments, an elongated septum 1432a extending from the diaphragm 1432 of the flexible valve element 1430 can extend into the clearance defined between the housing 1402a and the collar 1402b. As illustrated, the diameter of the 1402b can be reduced to permit clearance for the septum 1432a within the housing 1402.

As illustrated, the upper portion of the collar 1402b1 can be separate or dis-integrated from the lower portion or base 1402b2 to allow for modularity or configurability of the needleless connector 1400.

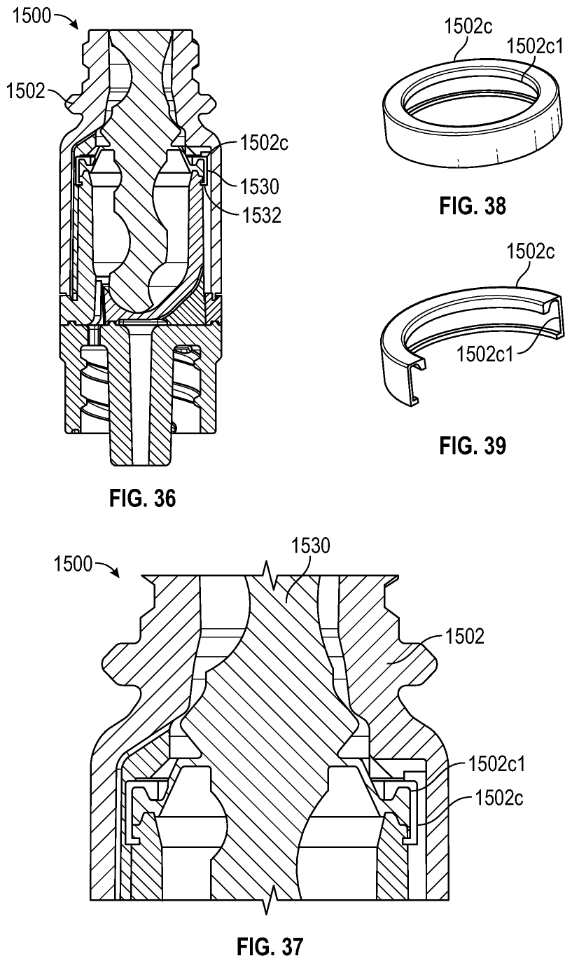

FIG. 36 is a cross-sectional view of a needleless connector 1500, in accordance with various aspects of the present disclosure. FIG. 37 is a detail cross-sectional view of a needleless connector 1500, in accordance with various aspects of the present disclosure. FIG. 38 is a perspective view of a collar 1502c, in accordance with various aspects of the present disclosure. FIG. 39 is a partial cross-sectional view of a collar 1502c, in accordance with various aspects of the present disclosure. In some embodiments, the needleless connector 1500 can include features that are similar to needleless connector 200. Accordingly, certain features of the needleless connector 1500 can be referred to with similar reference numerals.

In the depicted example, the housing 1502 can retain or capture the flexible valve element 1530 relative to the housing 1502 to maintain sterility of the fluid path and maintain the position of the diaphragm 1532 relative to the housing 1502. In some embodiments, the housing 1502 can include a collar or snap ring 1502c to capture and retain the diaphragm 1532 of the flexible valve element 1530. As illustrated, the diaphragm 1532 can be captured in the channel 1502c1 of the snap ring 1502c. In some embodiments, fluid can form a liquid column above the diaphragm 1532 and air can form an air column below the diaphragm 1532.

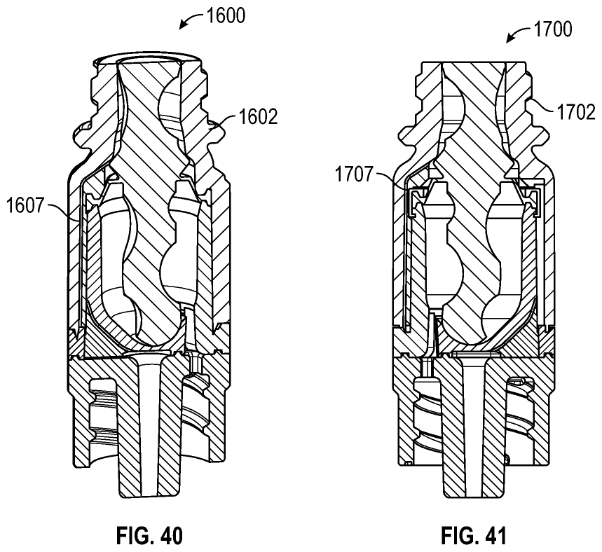

FIG. 40 is a cross-sectional view of a needleless connector 1600, in accordance with various aspects of the present disclosure. In some embodiments, the needleless connector 1600 can include features that are similar to needleless connector 100. Accordingly, certain features of the needleless connector 1600 can be referred to with similar reference numerals.

In the depicted example, the housing 1602 can define one or more flow channels 1607 to facilitate flushing of the needleless connector 1600. In some embodiments, the housing 1602 can define a single flow channel 1607 with a support ring. Optionally, the housing 1602 and the support ring can define multiple flow channels.

FIG. 41 is a cross-sectional view of a needleless connector 1700, in accordance with various aspects of the present disclosure. In some embodiments, the needleless connector 1700 can include features that are similar to needleless connector 100. Accordingly, certain features of the needleless connector 1700 can be referred to with similar reference numerals.

In the depicted example, the housing 1702 can define multiple, smaller flow channels 1707 to facilitate flushing of the needleless connector 1700. In some embodiments, the housing 1702 can define the flow channels 1707 without the use of a support ring.

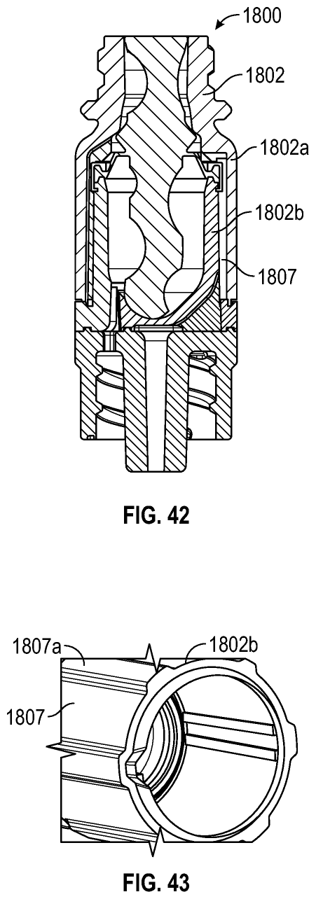

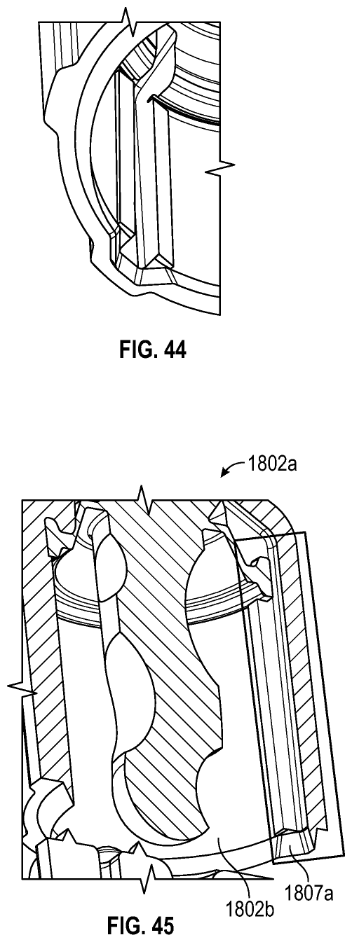

FIG. 42 is a cross-sectional view of a needleless connector 1800, in accordance with various aspects of the present disclosure. FIG. 43 is a detail view of a housing portion 1802b, in accordance with various aspects of the present disclosure. FIG. 44 is a detail view of a housing portion 1802b, in accordance with various aspects of the present disclosure. FIG. 45 is a detail cross-sectional view of a needleless connector 1800, in accordance with various aspects of the present disclosure. In some embodiments, the needleless connector 1800 can include features that are similar to needleless connector 100. Accordingly, certain features of the needleless connector 1800 can be referred to with similar reference numerals.

In the depicted example, the housing 1802 can define one or more flow channels 1807 to facilitate flushing of the needleless connector 1800. In some embodiments, the housing 1002 is a two-part housing that defines the flow channels 1807 between the two parts of the housing 1802. As illustrated, the flow channels 1807 can be defined between an outer portion 1802a and an inner portion 1802b of the housing. In some embodiments, the flow channels 1807 can be defined and/or contained by crush ribs 1807a extending from the surface of the inner portion 1802b to prevent the weeping of fluid to other portions or outside of the housing 1802.

As illustrated, the crush ribs 1807a can be molded on a portion of the inner housing 1802b and engage with (via interference fit) with the interior geometry of the outer housing 1802a to form liquid-tight barriers. In some embodiments, fluid can form a liquid column above the within the flow channels 1807.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A needleless connector, comprising:
a housing comprising a cavity, a proximal fluid port in fluid communication with the cavity, and a distal fluid port in fluid communication with the cavity; and
a flexible valve element disposed within the cavity to selectively permit flow between the proximal fluid port and the distal fluid port.

2. The needleless connector of claim 1, wherein the flexible valve element comprises a valve diaphragm, and the valve diaphragm is configured to expand when a luer is inserted into the proximal fluid port and retract when the luer is removed from the proximal fluid port.

3. The needleless connector of claim 2, wherein the flexible valve element comprises a cone coupled to the valve diaphragm and extending toward the proximal fluid port.

4. The needleless connector of claim 2, wherein the flexible valve element comprises a shroud coupled to the valve diaphragm and extending toward the distal fluid port.

5. The needleless connector of claim 2, wherein the valve diaphragm defines a flat portion.

6. The needleless connector of claim 2, wherein the valve diaphragm is asymmetrical.

7. The needleless connector of claim 2, wherein the valve diaphragm comprises a retention edge, wherein at least a portion of the retention edge is captured within the housing.

8. The needleless connector of claim 7, wherein the retention edge is deformed within the housing.

9. The needleless connector of claim 1, wherein the flexible valve element comprises a valve tail extending into the cavity, wherein the valve tail deflects to selectively permit flow between the proximal fluid port and the distal fluid port.

10. The needleless connector of claim 9, wherein the valve tail comprises a plurality of bends.

11. The needleless connector of claim 9, wherein the valve tail is configured to minimize insertion force of a luer to permit flow between the proximal fluid port and the distal fluid port.

12. The needleless connector of claim 1, wherein the housing comprises a proximal portion and a distal portion, wherein the proximal portion and the distal portion are coupled together.

13. The needleless connector of claim 12, wherein at least one of the proximal portion and the distal portion comprises polypropylene or polyethylene.

14. The needleless connector of claim 12, wherein at least one of the proximal portion and the distal portion comprises polycarbonate or copolyester.

15. The needleless connector of claim 12, wherein the proximal portion and the distal portion are coupled via press fit or snap fit.

16. The needleless connector of claim 12, wherein the proximal portion and the distal portion are coupled via adhesive or ultrasonic welding.

17. The needleless connector of claim 12, further comprising a snap ring configured to couple the proximal portion and the distal portion.

18. The needleless connector of claim 1, wherein the housing defines at least one flow channel in fluid communication with the proximal fluid port and the distal fluid port.

19. The needleless connector of claim 1, wherein the distal fluid port is laterally offset relative to the proximal fluid port.

20. A needleless connector, comprising:
a housing comprising:
a proximal portion;
a distal portion, wherein the proximal portion and the distal portion are coupled together to cooperatively define a cavity;
a proximal fluid port in fluid communication with the cavity; and
a distal fluid port in fluid communication with the cavity; and
a flexible valve element comprising:
a valve diaphragm, and the valve diaphragm is configured to expand when a luer is inserted into the proximal fluid port and retract when the luer is removed from the proximal fluid port;
a cone coupled to the valve diaphragm and extending toward the proximal fluid port;
a retention edge, wherein at least a portion of the retention edge is captured within the housing; and
a valve tail extending into the cavity, wherein the valve tail deflects to selectively permit flow between the proximal fluid port and the distal fluid port.
